(19) United States
(12) Patent Application Publication
(10) Pub. No.: US 2019/0150880 A1
(43) Pub. Date: May 23, 2019

(54) Autoclavable Stethoscope Assembly

(71) Applicants: Cameron Campbell, Cary, NC (US); Alexis Campbell, Cary, NC (US)

(72) Inventors: Cameron Campbell, Cary, NC (US); Alexis Campbell, Cary, NC (US)

(21) Appl. No.: 15/820,412

(22) Filed: Nov. 21, 2017

(51) Int. Cl. A61B 7/02; A61L 2/04; G10K 11/08

(52) U.S. Cl. CPC A61B 7/02; A61L 2202/24; G10K 11/08; A61L 2/04

(57) ABSTRACT

An autoclavable stethoscope assembly for facilitating a stethoscope to be sterilized in an autoclave includes a cup that is selectively positioned against an individual thereby facilitating the cup to detect audible sounds within the individual. The cup is comprised of a heat resistant material for sterilizing in an autoclave. A bell diaphragm is removably coupled to the cup and the bell diaphragm abuts the individual when the cup is positioned against the individual. The bell diaphragm is comprised of a tympanic material to amplify the audible sounds in the individual. A grip is coupled to the cup and the grip is selectively gripped for manipulating the cup. The grip is comprised of a heat resistant material for sterilizing in an autoclave.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to autoclavable stethoscope assemblies and more particularly pertains to a new autoclavable stethoscope assembly for facilitating a stethoscope to be sterilized in an autoclave.

### BRIEF SUMMARY OF THE INVENTION

An embodiment of the disclosure meets the needs presented above by generally comprising a cup that is selectively positioned against an individual thereby facilitating the cup to detect audible sounds within the individual. The cup is comprised of a heat resistant material for sterilizing in an autoclave. A bell diaphragm is removably coupled to the cup and the bell diaphragm abuts the individual when the cup is positioned against the individual. The bell diaphragm is comprised of a tympanic material to amplify the audible sounds in the individual. A grip is coupled to the cup and the grip is selectively gripped for manipulating the cup. The grip is comprised of a heat resistant material for sterilizing in an autoclave.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of an autoclavable stethoscope assembly according to an embodiment of the disclosure.

FIG. 2 is a bottom view of an embodiment of the disclosure.

FIG. 3 is a right side view of an embodiment of the disclosure.

FIG. 4 is a top view of an embodiment of the disclosure.

### DETAILED DESCRIPTION OF THE INVENTION

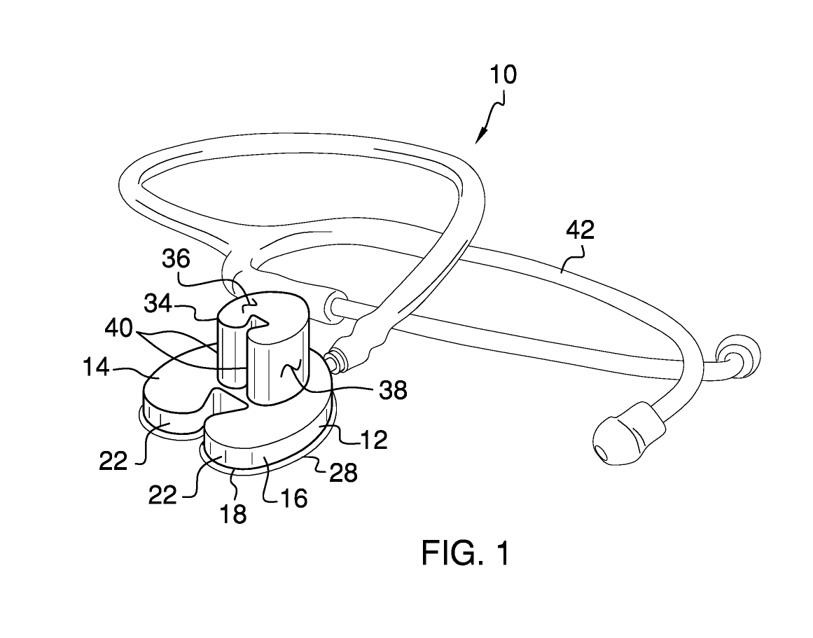

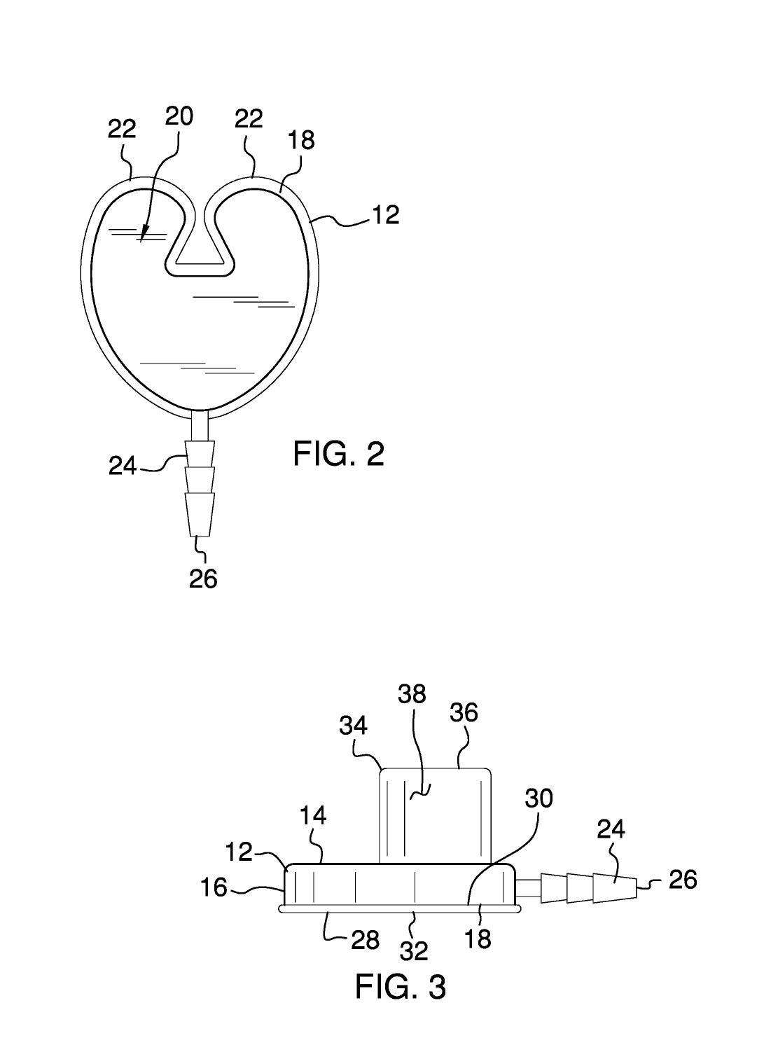

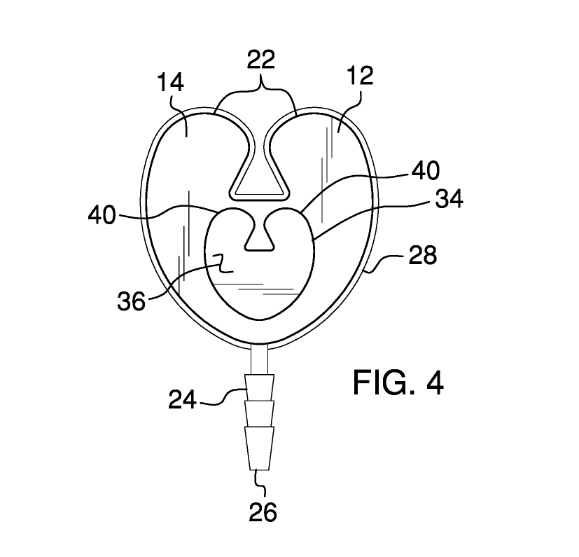

With reference now to the drawings, and in particular to FIGS. 1 through 4 thereof, a new autoclavable stethoscope assembly embodying the principles and concepts of an embodiment of the disclosure and generally designated by the reference numeral 10 will be described.

As best illustrated in FIGS. 1 through 4, the autoclavable stethoscope assembly 10 generally comprises a cup 12 that is selectively positioned against an individual thereby facilitating the cup 12 to detect audible sounds within the individual. The audible sounds within the individual may be sounds associated with heartbeat, respiration, digestion and any other biological processes occurring within the individual. The individual may be a patient in a medical care environment. The cup 12 is comprised of a heat resistant material such as ceramic or the like such that the cup 12 may be sterilized in an autoclave.

The cup 12 has a top wall 14 and a perimeter wall 16 extending away therefrom and the perimeter wall 16 is coextensive with a perimeter of the top wall 14. The perimeter wall 16 has a distal edge 18 with respect to the top wall 14 to define an opening 20 into the cup 12 and the opening 20 passes the audible sound through the cup 12. The distal edge 18 abuts the individual when the cup 12 is positioned on the individual. Moreover, the perimeter wall 16 is continuous such that the cup 12 has a circular cross section taken along a line extending through the top wall 14 and the distal edge 18 of the perimeter wall 16.

The perimeter wall 16 curves inwardly toward a center of the top wall 14 to define a pair of first lobes 22 on the perimeter wall 16. The first lobes 22 are spaced apart from each other and each of the first lobes 22 enhances gripping the cup 12. The pair of first lobes 22 facilitates a right handed user and a left handed user to grip the cup 12. A stem 24 is coupled to and extends away from the perimeter wall 16 of the cup 12. The stem 24 is substantially hollow such that the stem 24 is in fluid communication with the cup 12 to pass the audible sounds out of the cup 12. The stem 24 has a distal end 26 with respect to the cup 12 and the distal end 26 is open to pass the audible sounds out of the stem 24.

A bell diaphragm 28 is provided and the bell diaphragm 28 is removably coupled to the cup 12. The bell diaphragm 28 abuts the individual when the cup 12 is positioned against the individual. Moreover, the bell diaphragm 28 is comprised of a tympanic material to amplify the audible sounds in the individual. The bell diaphragm 28 has a first surface 30 and a second surface 32 and the first surface 30 is removably attached to the distal edge 18 of the perimeter wall 16 of the cup 12. The second surface 32 abuts the individual when the cup 12 is positioned against the individual. The bell diaphragm 28 may be removably retained on the cup 12 through any conventional mechanical means, including but not being limited to, an adhesive layer and tabs and grooves.

A grip 34 is coupled to the cup 12 and the grip 34 is gripped thereby enhancing manipulating the cup 12. The grip 34 is comprised of a heat resistant material, such as ceramic or the like, thereby facilitating the grip 34 to be sterilized in an autoclave. The grip 34 extends away from the top wall 14. The grip 34 has a distal surface 36 with respect to the top wall 14 and a peripheral surface 38 extending between the distal surface 36 and the top wall 14. The peripheral surface 38 is spaced from the perimeter wall 16 of the cup 12. Moreover, the peripheral surface 38 is coextensive with the perimeter wall 16 such that the peripheral surface 38 defines a pair of second lobes 40 on the grip 34. Each of the second lobes 40 enhances gripping the grip 34 for the right handed user and the left handed user.

A pair of earbuds 42 is provided and each of the earbuds 42 is removably coupled to the stem 24. Each of the earbuds 42 is in fluid communication with the bell diaphragm 28 to communicate the audible sounds within the individual. The earbuds 42 are selectively positioned in a user's ears thereby facilitating the user to hear the audible sounds within the individual. The earbuds 42 may be stethoscope earbuds of any conventional design.

In use, the bell diaphragm 28 is attached to the cup 12 prior to placing the cup 12 against the individual. The bell diaphragm 28 abuts the individual when the cup 12 is placed against the individual thereby facilitating the user to hear the audible sounds within the individual. The bell diaphragm 28 and the ear buds are removed from the cup 12 after the cup 12 is no longer being positioned against the individual. The cup 12 and the grip 34 are placed in an autoclave for sterilizing the cup 12 and the grip 34 thereby reducing the transmission of communicable diseases via the cup 12 and the grip 34. In this way cleanliness and sterility of a medical environment, such as a hospital or nursing home, is enhanced with respect to transferring bacteria between individuals and between individuals and the user.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An autoclavable stethoscope assembly being configured to be sterilized with intense heat, said assembly comprising:
a cup being configured to be positioned against an individual thereby facilitating the cup to detect audible sounds within the individual, said cup being comprised of a heat resistant material wherein said cup is configured to be sterilized in an autoclave;
a bell diaphragm being removably coupled to said cup wherein said bell diaphragm is configured to abut the individual when said cup is positioned against the individual, said bell diaphragm being comprised of a tympanic material wherein said bell diaphragm is configured to amplify the audible sounds in the individual; and
a grip being coupled to said cup wherein said grip is configured to be selectively gripped, said grip being comprised of a heat resistant material wherein said grip is configured to be sterilized in an autoclave.

2. The assembly according to claim 1, wherein said cup has a top wall and a perimeter wall extending away therefrom, said perimeter wall being coextensive with a perimeter of said top wall, said perimeter wall having a distal edge with respect to said top wall to define an opening into said cup wherein said opening is configured to pass the audible sound through said cup, said distal edge being configured to be positioned against the individual, said perimeter wall is continuous such that said cup has a circular cross section taken along a line extending through said top wall and said distal edge of said perimeter wall.

3. The assembly according to claim 2, wherein said perimeter wall curves inwardly toward a center of said top wall to define a pair of first lobes on said perimeter wall, said first lobes being spaced apart from each other wherein each of said first lobes is configured to enhance gripping said cup.

4. The assembly according to claim 2, further comprising a stem being coupled to and extending away from said perimeter wall of said cup, said stem being substantially hollow such that said stem is in fluid communication with said cup wherein said stem is configured to pass the audible sounds out of said cup.

5. The assembly according to claim 2, wherein said bell diaphragm has a first surface and a second surface, said first surface being removably attached to said distal edge of said perimeter wall of said cup, said second surface being configured to abut the individual when said cup is positioned against the individual.

6. The assembly according to claim 2, wherein said grip extends away from said top wall, said grip having a distal surface with respect to said top wall and a peripheral surface extending between said distal surface and said top wall, said peripheral surface being spaced from said perimeter wall of said cup.

7. The assembly according to claim 6, wherein said peripheral surface is coextensive with said perimeter wall such that said peripheral surface defines a pair of second lobes on said grip wherein each of said second lobes is configured to enhance gripping said grip.

8. The assembly according to claim 2, further comprising a pair of earbuds, each of said earbuds being removably coupled to said cup, each of said earbuds being in fluid communication with said bell diaphragm wherein each of said earbuds is configured to communicate the audible sounds within the individual, said earbuds being configured to be positioned in a user's ears thereby facilitating the user to hear the audible sounds within the individual.

9. An autoclavable stethoscope assembly being configured to be sterilized with intense heat, said assembly comprising:
a cup being configured to be positioned against an individual thereby facilitating the cup to detect audible sounds within the individual, said cup being comprised of a heat resistant material wherein said cup is configured to be sterilized in an autoclave, said cup having a top wall and a perimeter wall extending away therefrom, said perimeter wall being coextensive with a perimeter of said top wall, said perimeter wall having a distal edge with respect to said top wall to define an opening into said cup wherein said opening is configured to pass the audible sound through said cup, said distal edge being configured to be positioned against the individual, said perimeter wall being continuous such that said cup has a circular cross section taken along a line extending through said top wall and said distal edge of said perimeter wall, said perimeter wall curving inwardly toward a center of said top wall to define a pair of first lobes on said perimeter wall, said first lobes being spaced apart from each other wherein each of said first lobes is configured to enhance gripping said cup;
a stem being coupled to and extending away from said perimeter wall of said cup, said stem being substantially hollow such that said stem is in fluid communication with said cup wherein said stem is configured to pass the audible sounds out of said cup, said stem having a distal end with respect to said cup, said distal end being open wherein said distal end is configured to pass the audible sounds out of said stem;
a bell diaphragm being removably coupled to said cup wherein said bell diaphragm is configured to abut the individual when said cup is positioned against the individual, said bell diaphragm being comprised of a tympanic material wherein said bell diaphragm is configured to amplify the audible sounds in the individual, said bell diaphragm having a first surface and a second surface, said first surface being removably attached to said distal edge of said perimeter wall of said cup, said second surface being configured to abut the individual when said cup is positioned against the individual;
a grip being coupled to said cup wherein said grip is configured to be selectively gripped, said grip being comprised of a heat resistant material wherein said grip is configured to be sterilized in an autoclave, said grip extending away from said top wall, said grip having a distal surface with respect to said top wall and a peripheral surface extending between said distal surface and said top wall, said peripheral surface being spaced from said perimeter wall of said cup, said peripheral surface being coextensive with said perimeter wall such that said peripheral surface defines a pair of second lobes on said grip wherein each of said second lobes is configured to enhance gripping said grip; and
a pair of earbuds, each of said earbuds being removably coupled to said cup, each of said earbuds being in fluid communication with said bell diaphragm wherein each of said earbuds is configured to communicate the audible sounds within the individual, said earbuds being configured to be positioned in a user's ears thereby facilitating the user to hear the audible sounds within the individual.
